(19) United States
(12) Patent Application Publication
(10) Pub. No.: US 2024/0089678 A1
(43) Pub. Date: Mar. 14, 2024

(54) HEARING APPARATUS

(71) Applicant: Earswitch Ltd., Rutland (GB)

(72) Inventor: Nicholas Roy Gompertz, Rutland (GB)

(73) Assignee: Earswitch Ltd., Rutland (GB)

(21) Appl. No.: 18/274,804

(22) PCT Filed: Jan. 28, 2022
(86) PCT No.: PCT/GB2022/050226

§ 371 Date: Jul. 28, 2023

(30) Foreign Application Priority Data
Jan. 28, 2021 (GB) 2101131.7

(51) Int. Cl. H04R 25/00; A61F 11/04; H04R 17/00

(52) U.S. Cl. CPC H04R 25/606; A61F 11/045; H04R 17/005; H04R 25/353; H04R 25/356; H04R 2217/03; H04R 2225/67

(57) ABSTRACT

An apparatus for affecting pressure waves within at least part of or in proximity to an ear of a user. At least part of the apparatus is configured to be wearable within at least part of or in proximity to the ear of the user. The apparatus further includes a processor operative in response to an input signal of an input to control an output of an ultrasound emitter. The ultrasound emitter is configured to generate an ultrasound output which affects air and/or biological tissue in proximity to the ultrasound emitter.

### CROSS REFERENCE TO RELATED APPLICATIONS

This application is the U.S. National Phase Application of PCT International Application No. PCT/GB2022/050226, filed Jan. 28, 2022, which claims priority to Great Britain Patent Application No. GB 2101131.7, filed Jan. 28, 2021, the contents of such applications being incorporated by reference herein.

### FIELD OF THE INVENTION

The present invention relates to hearing apparatus, in particular an apparatus for affecting pressure waves within at least part of or in proximity to an ear of a user and an associated method, and a cochlear apparatus for implantation within at least part of a cochlear canal of a user or for replacement of a cochlea of a user.

### BACKGROUND OF THE INVENTION

Assistive hearing devices which are used to improve or enhance hearing, such as hearing-aids and smart earphones, generally transmit to the ear-drum an amplified version of sound received at the ear or ear-canal of a user. Users of these assistive hearing devices may, however, hear feedback sound because the audible sound frequencies emitted from the device are often transmitted not only to the ear-drum but also to an input microphone of the device which originally detects the sound received. Following that, many assistive hearing devices incorporate a structure that obstructs the ear-canal to ensure efficient propagation of the sound wave towards the ear-drum and to reduce propagation of the emitted sound to the microphone of the device—thereby reducing feedback. This is done so as to facilitate only amplification of specific frequencies or sounds. Unfortunately, this brings with it various disadvantages, which include obstruction of the ear-canal. Further owing to the typical size of the hearing devices cerumen (wax) in the external aspect of the ear-canal is pushed further into the ear-canal, which affects the normal active migration of ear-wax towards the external ear (pinna). Further disadvantageously, this effect can, over time, lead to increased ear-wax within the ear-canal that can block the emitter of the hearing device, and also block the ear-canal, causing reduced transmission of sounds and (further) reduced hearing. An obstructed ear-canal also reduces ventilation and increases humidity, providing an increased risk of inflammatory conditions such as otitis externa, which often causes pain, itching and discharge, and may render the assistive device uncomfortable to wear and further block the device or sound passage with discharge. In the alternative, assistive hearing devices which do not obstruct the ear-canal tend to allow external background noise to reach the ear-drum, which makes it difficult for the user to hear sounds of interest (such as speech).

Devices for transmitting other sounds such as music, telephone audio and any other sound, for example earphones or smart earphones, which will subsequently be termed ‘audio device(s)’, may include a function to reduce external sound that is transmitted to the ear-drum, for example by active noise cancellation. This function is improved when the ear-canal is obstructed by the audio device, so that the emitted sound from the audio device interferes with the external sound to cancel the amplitude of the external sound. In addition, some audio devices allow ‘pass through’ of some external sounds to the user's ear-drum, such as voice frequencies; however, they are not without their own disadvantages, which are similar to those in the paragraph above owing to obstruction.

Other similar devices provide active noise cancellation to protect the ear-drum and ear structures from excessive noise that can damage hearing. These devices also obstruct the ear-canal and, so, can interfere with the user hearing other significant sounds such as speech or warning sounds.

Developments in this field have led to the use of lasers to vibrate the ear-drum to create sound and reduce the risk of feedback. This has included using a contact hearing system and/or tympanic membrane transducer (sometimes referred to as a ‘lens’) positioned against the ear-drum to efficiently transmit the laser signal to the ear-drum. This, of course, initially requires the intervention of a healthcare worker to position the lens, and it must be in line of sight of the laser emitter. Further, this lens needs to be retained in a position against the ear-drum and, so, may damage or irritate the ear-drum, and may move relative to the laser emitter owing to gravity on changing body position, sneezing, coughing, etc., and may reduce air conduction of normal external sounds.

For those who have significantly impaired hearing, a cochlear implant may be used to provide an amount of hearing efficacy. Cochlear implants rely on external microphones and tend to rely upon wired connections to the implant. Disadvantages include a risk of infection and other complications from a wired connection, and the detection by the external microphones of unwanted background noise.

Descriptions of ultrasound induced bone conduction hearing aids are limited by the use of microphones external to the ear that amplify unwanted background noise and require a hearing device with firm contact with the bone of the skull through externally located emitters. These clearly operate very differently from the proposed invention.

There is, therefore, a need for improved hearing apparatus.

### SUMMARY OF THE INVENTION

According to a first aspect, the present invention provides an apparatus for affecting (and/or providing) pressure waves within at least part of or in proximity to an ear of a user, at least part of the apparatus being configured to be wearable within at least part of or in proximity to said ear of said user, the apparatus further comprises processor means operative in response to an input signal of an input means to control an output of an ultrasound emitter, wherein the ultrasound emitter is configured to generate an ultrasound output which affects air and/or biological tissue in proximity to the ultrasound emitter.

Preferably, the ultrasound output is configured to be capable of transmitting, either directly or indirectly, ultrasound towards one or more of the group comprising:
a) at least part of:
   i) an ear-drum complex to affect movement and/or vibration of at least part of the ear-drum complex; or
   ii) an inner and/or middle ear structure or space(s) to affect movement and/or vibration of at least part of an inner and/or middle ear structure or space(s);
b) at least part of an ear-canal, to affect movement and/or vibration of at least part of an ear-canal wall, adjacent bone and/or volume of air in the ear-canal;
c) at least part of a cochlear implant or hearing prosthetic, to affect movement and/or vibration of at least part of the cochlear implant or hearing prosthetic;
d) at least part of an external opening of an/the ear canal,
e) a region adjacent an outer ear of said user; and/or
f) a region adjacent, or radially inwards of, the apparatus.

Preferably, the ear-drum complex comprises the ear-drum and/or the malleus; most preferably, it comprises the ear-drum and malleus.

Preferably, the ultrasound output is transmitted to at least part of said ear-drum complex, inner or middle ear, ear-canal or other structure or space(s), or to at least part of said cochlear implant or hearing prosthetic to affect or provide a perception of sound to said user.

Preferably, the ultrasound output transmitted to at least part of said ear-canal, said region adjacent said outer ear of said user, or said region adjacent, or radially inwards of, the apparatus is configured to reduce or obstruct the effects of an adverse pressure wave reaching said ear-drum complex, cochlear implant or hearing prosthetic.

Preferably, the ultrasound output generates a fluctuating (air) pressure wave of variable frequency and/or amplitude configured to replicate vibration of an ear-drum commensurate with vibrations expected in a normal ear in response to sound.

Preferably, the ultrasound output generates one or more of the group comprising:
a pressure effect at or within, or in proximity to an ear, ear-drum and/or ear-canal; patterns of ultrasound waves that interfere at one or many points, areas and/or two- or three-dimensional planes to affect a change in pressure at or close to any of the points, areas or planes;
patterns of ultrasound waves that interfere at a position or area, in or adjacent to a site or position within the ear-canal, the external auditory meatus, and/or the ear-canal wall and/or surrounding bone;
a constant or fluctuating pressure wave of constant or varying frequency and/or amplitude configured to provide interference to reduce or obstruct the effects of an adverse pressure wave reaching said ear-drum complex, cochlear implant or hearing prosthetic;
a constant pressure wave configured to act as an acoustic filter or acoustic grate, to reduce or prevent transmission of specific frequencies and/or amplitudes of an adverse pressure wave reaching said ear-drum complex, cochlear implant or hearing prosthetic; and/or
patterns of ultrasound waves to provide interference to reduce or obstruct the effects of an adverse pressure wave reaching said ear-drum complex, cochlear implant or hearing prosthetic, or modify an adverse pressure wave to remove its adverse effect(s).

Preferably, the input means comprises:
a control signal;
a sound input; and/or
a detector, for detecting sound or changes in pressure at or near said ear of said user.

Preferably, the input signal to the processor means is analysed to determine frequencies and corresponding amplitudes of the sound input signal and/or detected sound signal, and the processor means provides an output signal for the emitter means which is configured to affect:
at least part of the cochlear implant or hearing prosthetic to generate signals in the auditory nerve which are perceived by said user as audible sound; or
the ear-drum complex consistent with said user hearing the sound input or detected sound.

Preferably, a distance along the cochlear implant where an electrical current is generated determines a frequency of audible sound perceived by said user, and is itself determined by frequency, amplitude and/or modulation of the ultrasound output.

Preferably, the emitter means is configured to affect vibration and/or movement of:
a) ear structures, to include but not limited to a part or parts of the ear-drum, malleus, incus, stapes, middle ear muscle (which may include the tensor tympani), oval window, round window, or membranes thereof, cochlea or adjacent bone, semi-circular canals, ear-canal wall and/or bones surrounding the ear-canal wall, or within fluid of the cochlea or middle ear; OR
b) artificial ear structures, to include but not limited to a part or parts of a cochlear implant, hearing prosthetic, and/or inner or outer ear structure, or artificial ear structures intended to replace any of those in a) above.

Preferably, the processor means is configured to provide an output signal to the ultrasound emitter for providing ultrasound of predetermined amplitude, frequency, duration, modulation, harmonics, timing, and/or interference pattern.

Preferably, the processor means is configured to additionally analyse one or more inputs from an ultrasound transducer/receiver, a laser and/or light emitter/receiver, microphone, proximity sensor, a time of flight sensor, an imager, and/or an ultrasound transducer/receiver, or combination thereof, which detect movement and/or vibration of any ear structure, or a movement in close proximity to said ear, and, thereby, modify the output signal.

Preferably, the processor means is configured to compare a detected response of one or more ear structures to the ultrasound output with an expected response to sound, and is configured to modify the ultrasound output to improve replication of the sound.

Preferably, the processor means is configured to provide an output signal which actively reduces or interferes with non-preferred sound at the ear-drum, so as to enhance perception of preferred sound by said user.

Preferably, one or more sensors of the apparatus are capable of detecting movement of the ear-drum and/or ear structures corresponding to: voluntary ear-drum movement; physiological eardrum movement or changes; eye-movement or other facial movement; movement related to auditory attention; and/or a sound heard by a user, and the processor means is capable of modifying the output signal.

Preferably, the ultrasound emitter is configured to be located within at least part of or in proximity to said ear of said user; preferably within or around the ear-canal. Preferably, the apparatus is configured to be located in or around an ear canal of said user.

Preferably, the apparatus further comprises a cochlear apparatus according to the third aspect.

According to a second aspect, the present invention provides a method for affecting (and/or providing) pressure waves within at least part of or in proximity to an ear of a user, the method comprises controlling an output of an ultrasound emitter in response to an input signal of an input means,
wherein the ultrasound emitter is worn within at least part of or in proximity to the ear of the user and generates an ultrasound output which affects air and/or biological tissue of the user in proximity to the ultrasound emitter.

Preferably, the pressure waves are created or modified by the ultrasound output. Preferably, the ultrasound output transmits, either directly or indirectly, ultrasound towards one or more of the group comprising:
a) at least part of
   i) an ear-drum complex of the user to affect movement and/or vibration of at least part of the ear-drum complex; or
   ii) an inner and/or middle ear structure or space(s) to affect movement and/or vibration of at least part of an inner and/or middle ear structure or space(s);
b) at least part of an ear-canal of the user, to affect movement and/or vibration of at least part of an ear-canal wall, adjacent bone and/or volume of air in the ear-canal;
c) at least part of a cochlear implant or hearing prosthetic of the user, to affect movement and/or vibration of at least part of the cochlear implant or hearing prosthetic;
d) at least part of an external opening of an/the ear-canal,
e) a region adjacent an outer ear of the user; and/or
f) a region adjacent, or radially inwards of, an apparatus.

Preferably, the ultrasound output transmits to at least part of the ear-drum complex, inner or middle ear, ear-canal or other structure or space, or to at least part of the cochlear implant or hearing prosthetic to provide a perception of sound to the user.

Preferably, the ultrasound output transmits to at least part of the ear-canal, the region adjacent the outer ear of the user, or the region adjacent, or radially inwards of, the apparatus to reduce or obstruct the effects of an adverse pressure wave reaching said ear-drum complex, cochlear implant or hearing prosthetic.

Preferably, the ultrasound output generates a fluctuating pressure wave of variable frequency and/or amplitude for replicating vibration of an/the ear-drum commensurate with vibrations expected in a normal ear in response to sound.

Preferably, the ultrasound output generates one or more of the group comprising:
a pressure effect at or within, or in proximity to an ear, ear-drum and/or ear-canal;
patterns of ultrasound waves that interfere at one or many points, areas and/or two- or three-dimensional planes to affect a change in pressure at or close to any of the points, areas or planes;
patterns of ultrasound waves that interfere at a position or area, in or adjacent to a site or position within the ear-canal, the external auditory meatus, and/or the ear-canal wall and/or surrounding bone;
a constant or fluctuating pressure wave of constant or varying frequency and/or amplitude for providing interference to reduce or obstruct the effects of an/the adverse pressure wave reaching said ear-drum complex, cochlear implant or hearing prosthetic;
a constant pressure wave for acting as an acoustic filter or acoustic grate, to reduce or prevent transmission of specific frequencies and/or amplitudes of an adverse pressure wave reaching said ear-drum complex, cochlear implant or hearing prosthetic; and/or
patterns of ultrasound waves for providing interference to reduce or obstruct the effects of an adverse pressure wave reaching said ear-drum complex, cochlear implant or hearing prosthetic, or modify an adverse pressure wave to remove its adverse effects.

Preferably, an input signal to a processor means is analysed to determine frequencies and corresponding amplitudes of sound input signal and/or detected sound signal, and the processor means provides an output signal for the emitter means which is configured to affect:
at least part of the cochlear implant or hearing prosthetic to generate signals in the auditory nerve which are perceived by the user as audible sound; or
the ear-drum complex consistent with the user hearing the sound input or detected sound.

Preferably, processing an input signal to compare a detected response of one or more ear structures to the ultrasound output with an expected response to sound, and modifying the ultrasound output to improve replication of the sound.

According to a third aspect, the present invention provides a cochlear apparatus, for implantation within at least part of a cochlear canal of a user or for replacement of a cochlea of a user, the apparatus comprises one or more regions adapted to receive an ultrasound output of an ultrasound emitter locatable within at least part of or in proximity to an ear of said user so as to generate one or more electrical signals for providing a perception of sound to said user.

Preferably, the apparatus comprises means for stimulating auditory nerve cells of the inner ear.

Preferably, the one or more regions comprise piezoelectric material capable of receiving said ultrasound output and generating one or more electrical signals.

Preferably, the one or more regions are spatially located along at least part of the apparatus such that different regions are responsive to different frequencies, amplitudes and/or modulation of said ultrasound output.

Preferably, the apparatus comprises a receiver for propagating received sound, ultrasound or electromagnetic signals to the cochlear apparatus.

Preferably, said ultrasound output agitates the one or more regions to generate electrical signals or other transfer of energy takes place, for example transfer of heat energy.

Preferably, a distance along the cochlear apparatus where an electrical current is generated determines a frequency of audible sound perceived by said user, and is itself determined by frequency, amplitude and/or modulation of said ultrasound output.

The present invention provides a method and apparatus for ultrasound emitters and receivers within a hearing assistive device, or other earphone, ear protection or other ear-related device (collectively referred to as ‘earphone(s)’), for transmitting and/or obstructing sound towards ear structures and/or a cochlear implant, preferably providing remote auditory attention monitoring and hands-free control. This and above aspects of the present invention may comprise any one or more of the following.

Preferably, transmitting and/or obstructing sound towards ear structures, or cochlear implants is conducted, either directly or indirectly, and provides efficient assistive hearing, sound transmission, and sound protection devices.

Preferably, the emitter comprises one or more emitters which are configured to form an array of emitters, preferably over a small area such as a micro-electromechanical system (MEMS) emitter or capacitive micro-machined ultrasonic transducer (CMUT). Preferably, the emitter is positioned in any direction including largely towards the ear-drum, largely towards the external auditory meatus, and/or towards any part of the ear-canal. Preferably, the output of each emitter is configured relative to an input sound to vary the frequency, amplitude and/or modulation of the ultrasound waves, so as to interfere with the output of all or any of the other emitters. Interference waves and/or patterns, or emitter output is/are, preferably, configured to be a defined pattern of interference at the position of the ear structures, for example ultrasound waves may be focused to interfere at a position of the ear-drum, forming pressure at a site of focus which varies in relation to the input sound.

Preferably, the emitter is configured to provide ultrasound waves of a frequency which vibrates the ear-drum, any other ear structures including any or all of the ossicular bones, membranes of the round and/or oval windows of the cochlea, and/or any part of the cochlea and/or adjacent bone, which can be collectively referred to as ‘ear structures’. Further preferably, the emitter is configured to provide ultrasound waves of a frequency which vibrates an implant device for assistive hearing, which includes a cochlear implant or other hearing prosthesis, or an interface including a device positioned adjacent the ear-drum, which can be collectively referred to as a ‘cochlear implant or hearing prosthetic’. Preferably, the emitter is a single emitter emitting a variable and/or constant ultrasound frequency and/or amplitude of ultrasound output, relative to the input sound to be transmitted to the ear structures. The emitter may transmit, and/or amplify and/or cancel sounds input from the device, by effecting vibration of ear structures.

Preferably, where multiple emitters are provided, the processor and algorithm are configured to generate independent ultrasound waves for one or more emitters, or each emitter. The independent ultrasound waves focus interference patterns at, or adjacent to, ear structures, or at a distance from the emitter(s) to affect movement of the ear structures in relationship to the sound sensed by the microphone and, thereby, transmit the audible sound to the user's ear structures by inaudible ultrasound signals.

Most preferably, a resultant vibration of the ear-drum is transmitted to the ossicular bones and the cochlea to be perceived by the user as sound.

Preferably, a distance at which the interference is focused may be fixed, for example to be at a set distance away from the earphone, or may be variable and alterable by the algorithm of the processor which receives an input from a sensor that detects a distance from the earphone to the ear-drum when worn.

Preferably, transmission of vibrations to the ear structure (for example the ear-drum) may be enhanced by the application of a substance such as a liquid, to include olive oil, mineral oil or similar liquid medium, which contains particles which enhance the transmission or reflection of emitted ultrasound waves.

Preferably, the ultrasound interference pattern may be focused to interfere at a location within, or adjacent, the ear-canal, or towards the external auditory meatus and/or the ear-canal wall or surrounding bone.

Preferably, the input to the processor is a sound input from one or more microphones, or an input external to the earphone. Sound detected by the microphone is transmitted as an input to the processor, and an algorithm of the processor generates an output to activate and control the ultrasound emitter. Preferably, the microphone, processor and/or algorithm are configured so that any received ultrasound is not amplified, which helps reduce audible feedback. Preferably, the audio device has one or more input microphones (or sensors), and functions additionally as an assistive hearing device, and/or functions to transmit ambient sound, which is termed ‘audio pass through’.

Preferably, the emitter(s) may generate ultrasound waves relative to sound input signals from any sound producing and/or transmitting electronic device, to include but not limited to an earphone, telephone speaker, music device, communication device, or any other device emitting sound signals or data, collectively referred to as ‘audio device’ or ‘audio input’. Preferably, an audio signal or sound signal from an audio device is transmitted as an input to a processor, and the algorithm generates an output to activate and control the ultrasound emitter.

Preferably, emitted ultrasound affects changes in assistive hearing devices, including cochlear implants, cochlear implant controllers, cochlear implant interfaces, or any other hearing prosthesis or interface including devices positioned adjacent the ear-drum. Preferably the ultrasound emitted from the earphone affects a change in the cochlear implant, hearing prosthesis, their associated interfaces and/or controllers, and provides a perception of sound for the user.

Preferably, a cochlear implant or hearing prosthesis requires no external device, wired interface, or internal wired connection between a controller and the cochlear implant, unlike in pre-existing cochlear implants. Preferably, a receiver of the cochlear implant or hearing prosthesis is positioned near to, or in contact with, the ear-drum, one or more ossicles, the round window membrane, and/or the oval window membrane. The receiver may also be positioned at the oval window which may have been surgically removed or breached, the round window, or in any position from or within a surgically formed boney canal close to the cochlea. Preferably, the receiver is within at least part of the cochlear canal, positioned behind (proximal) to the intact oval window or the intact round window. Alternatively, the cochlea may be a prosthetic or replacement cochlear device.

Preferably, the cochlear implant is positioned within the cochlear canal without requirement for a wired external power source. The energy requirement to generate auditory nerve signals may be met by wireless electromagnetic transmission, including wireless charging of an internal rechargeable battery, energy harvesting techniques related to body movements and/or temperature, and/or energy generation in association with received ultrasound emissions from the earphone. Preferably, a wireless cochlear implant having a receiving membrane, or interface of any material, transmits movement generated by the ultrasound to a piezoelectric material in the structure of the cochlear implant, the receiving membrane or interface generates electrical current of sufficient magnitude to charge the internal battery.

Preferably, the piezoelectric material generates electrical current which is supplied to a multitude of electrodes, arrays of electrodes, and/or electrical conducting materials positioned along a length of cochlear canal, such that the location of output of electrical current along the cochlear canal is dependent upon the received frequency and/or amplitude of the ultrasound, sound waves or signals received. Preferably, the cochlear implant comprises a structure, material or substance which is intended to be implanted in the cochlea (similarly to current cochlear implants) and which conducts a received sound or ultrasound wave to a position within the structure of the cochlear implant at a distance from the receiver which is relative to the frequency of the received sound or ultrasound waves.

Preferably, the cochlear implant is any solid, liquid, or combination of liquid and/or solid materials or substances. When liquid, it may be of any viscosity of liquid or combinations thereof in different locations within the cochlear implant. Preferably, conducted sound or ultrasound waves affect movement at a position of a structure of the cochlear implant such as between an internal structure and the external surface of piezoelectric material, which generates an electric current within the surface of the cochlear implant at a distance from the receiver relative to the received frequency of sound or ultrasound to stimulate adjacent nerve cells, which is perceived by the user as a frequency of sound relative to the frequency that was received by the microphone.

Preferably, in an alternative, the receiver, a controller, processor, and/or battery or energy generation device is located within the middle ear cavity, or external to the ear-drum in the ear-canal. Optionally, this could include a wired connection to the cochlear implant.

Preferably, the cochlear implant is fluid-filled and has an internal structure of one or a number of separate longitudinal chambers, or a different configuration, which may contain fluid of similar or different viscosities. Further preferably, the outer aspect of the chamber is located abutting, or close to, the nerve cells, and comprises electrical conduction material (such as electrodes) and/or separate or continuous piezoelectric materials which may be superficial, and/or extend into the structure of the cochlear implant. One end of the piezoelectric material may be fixed within a structure separating two or more chambers within the cochlear implant. Preferably, the cochlear implant may be located so as to have an outward-facing receiving surface—for example at the position of the oval or round window, which may have been surgically removed or disrupted, or within a surgically formed channel in continuity with the cochlea. Most preferably, the receiving surface propagates audio, ultrasound waves or electromagnetic signals to the material of the cochlear implant.

Preferably, ultrasound waves from the emitter affect pressure changes within the substance of the cochlear implant, which may be analogous to the frequency dependent pressure waves propagated at distances along a human cochlea that correspond to incident sound frequency. Most preferably, the pressure waves affect a movement of an aspect of the cochlear implant at a distance along the cochlear canal which generates an electrical current within the piezoelectric material that is adjacent to cochlear nerve cells (which may include the spiral ganglion neurones), and generates signals in the auditory (cochlea) nerve and are perceived by the user as sound. Most preferably, the distance along the cochlear implant where the wave is generated determines the frequency of the sound heard by the user.

Preferably, the microphone of the earphone, or other sound receiving device, sends audio signals to the processor, and the algorithm of the processor causes ultrasound to be emitted. Preferably, ultrasound is emitted from multiple emitters and interferes at a position within or close to the cochlear implant, to generate vibration at sound wave frequencies of the receiver, or generate pressure waves related to the input audio in the substance of the cochlear implant.

Preferably, the cochlear implant comprises at least partially piezoelectric material, or a similar material, which is stimulated to produce electrical current by the action of ultrasound

Preferably, the distance along the cochlear implant where electrical current is generated is determined by characteristics of the ultrasound emitted from ultrasound emitters of the earphone, or from ultrasound emitters which are located within or externally to the ear-canal, for example behind the ear or on the scalp.

Most preferably, the distance along the cochlear implant that the electrical current is generated is determined by the frequency, amplitude, and/or modulation of the ultrasound, which directly affects the electrical generation material or a distance a pressure wave moves along the cochlear implant.

Preferably, a structure that is an ultrasound receiver is positioned abutting or attached to ear structures, which can include the ear-drum, any combination of a single or multiple ossicles, middle ear muscles, oval or round window membranes or bone structures of the outer, middle or inner ear, where the structure enhances the receipt of the emitted ultrasound of the earphone and which enhances vibration of ear structures.

Preferably, in conjunction or separate from the above, an ultrasound emitter or emitters is directed towards a location or position largely external to the earphone such as within or close to the external auditory meatus, a position within the ear-canal more internal than the earphone but at a position external to the ear-drum, any aspect of the ear-canal wall, and/or radially inwards or outwards of the earphone towards the ear-canal walls.

Most preferably, the emitters are positioned largely in a direction external the auditory meatus, or radially. Most preferably, the earphone comprises a ring-shaped body including an internal aperture, or channel, in which is located one or more emitters. Preferably, the output of the emitters is configured to focus the ultrasound waves to interfere to cause an ultrasound standing wave (or positive interference pattern) within the external ear canal, within proximity to the external auditory meatus, towards the ear-drum but at an external distance from the ear-drum, laterally or radially around, or within the aperture or channel of the earphone.

Preferably, the ultrasound output is pre-configured to act as a barrier to external sounds (subsequently termed an ‘ultrasound barrier’) from entering into, or passing along the ear-canal. Preferably, the algorithm and/or processor varying the ultrasound output of the emitters, which has the effect of varying the resultant ultrasound interference patterns interfering with the external sounds, is subject to the input from the microphone.

Preferably, the earphone is user configurable, and/or adjustable, to enable control and adjustment of the level of obstruction provided by the ultrasound barrier.

Preferably, the ultrasound barrier acts to alter, reduce, and/or obstruct external sound entering into or passing along the ear-canal by acting as, but not limited to, a high-, low- or band-pass audio filter, an ultrasonic-, acoustic- and/or diffraction-grating. Preferably, this property is affected by a combination of frequencies/wavelengths/amplitudes and/or modulation of the ultrasound, any of amplitudes/frequencies or configuration of interference patterns or pressure waves, the latter of which includes, but is not limited to, inter-nodal spacing of interfering pressure waves, or largely parallel or circular pressure waves which act as gratings. Most preferably, speech sound frequencies may pass through the earphone to the ear-canal, whilst attenuating and/or deflecting away from the ear-canal lower frequency sounds such as background noise (e.g. in an aeroplane during flight).

Preferably, the emitters act as dual sensor and emitter(s), or sensors may separately detect a reflected signal from the ear-drum. Preferably, the sensor or sensors provide input to the processor and algorithm, and the algorithm receives input from a preferred audio signal (including but not limited to a microphone or an audio input such as music). The algorithm is configured to detect input signals from the ear-drum vibration and calculated ear-drum movement expected from the preferred audio signal. Most preferably, signals received from ear-drum movement that is at variance with that expected affects the output of the algorithm to emit ultrasound interference signals that prevent additional vibrations, actively cancelling noise at the ear-drum

Preferably, a dual emitter/sensor array emits wave patterns that interfere to focus upon the ear-drum of the user, causing different areas of the ear-drum to move by different amplitudes and frequencies according to the input sound signal.

Preferably, the single emitter or dual emitter/sensor arrays are located in or around a surface of the earphone, such that the arrays of emitters are aligned to direct largely to positions within the ear-canal, or internally within an aperture or channel of the earphone. Most preferably, the processor and algorithm receive input from the dual emitter/sensor arrays, other proximity or position sensors, gravity sensors, and/or accelerometers, and the output of the individual emitters within these arrays are configured to reflect the position of the emitter or arrays within the ear-canal. Most preferably, on positioning the earphone within the ear-canal, ultrasound is directed towards the ear-drum to transmit audio signals to the user, whilst those directed towards the external auditory meatus act as a barrier to external sound.

Preferably, when the earphone is next inserted by the user, different ultrasound sensors may be directed towards the ear-drum and the algorithm easily adapts to change the audio output to be emitted from those emitters previously acting as an ultrasound barrier, and vice versa. A spherical or similar shaped or three-dimensional earphone can thus be envisaged that could be placed within the ear-canal with no predefined position or orientation.

Preferably, ultrasound, or other sound or vibration is directed towards the ear-canal wall, which propagates sound signals through the adjacent bone to the middle and inner ear cavities.

Preferably, the ultrasound emitter(s) is/are configured for an individual user during use according to ultrasound signal (feedback) detected by emitters having dual emitter and receiver functions, or by a separate ultrasound receiver and/or microphone. Most preferably, detected feedback provides an input signal to the processor, and the algorithm alters the ultrasound output relative to that input signal.

Preferably, the method comprises the use of an emitter, or emitters, wholly or partially sited in or adjacent the ear canal, directed towards ear structures, such as the ear-drum complex (ear-drum and malleus), ear-canal wall, and/or external auditory meatus, or cochlear implant or prosthesis. Preferably, the emitter is located in a structure worn like a hearing aid, ear phone or earplug (subsequently referred to as earphone for convenience), or arm of eyeglass frame or head-set. Preferably, ultrasound emitters provide vibrations and movement of ear structures, such as the ear-drum complex, which is sensed by the user as a sound and is amplified sound (such as in a hearing-aid) or electronically transmitted sound (such as in an earphone or mobile phone, connected earphone), or affects changes in sound pressure in proximity to ear structures which stop or reduce (dampen) vibrations from ambient sound/noise (such as in passive or active noise cancellation headphones/earphones). Most preferably, there is a differential effect on the transmission (or reduction/dampening) of different frequencies or amplitudes of external sound. Most preferably, the emitted ultrasound waves generate effects of high-, low- or band-pass filters, and/or ultrasonic-, acoustic- or diffraction-gratings. Most preferably, the generated effects are provided by a pattern of interfering ultrasound waves of any combination of frequencies/wavelength/amplitude and/or modulation of ultrasound, and/or an amplitude/frequency or configuration of positive interference pattern or pressure wave. Preferably, these create a pattern of sound/air pressure waves, including different inter-nodal spacing of interfering pressure waves, largely parallel pressure waves, circular pressure waves, or other pattern of pressure waves which may act as acoustic, audio or ultrasonic gratings or filters.

Preferably, the processor, or processors, is located within the earphone, in proximity to the emitter, or is located externally and connected by wire or wirelessly. Preferably, the algorithm analyses sound input, such as input from a microphone or other electronic device like a mobile phone, music transmitting device or similar electronic device. Preferably, the algorithm provides an output to the emitter as a variable signal to effect a changing pressure wave positioned or focused at ear structures (such as the ear-drum). Preferably, analysis is individually configured according to the type of emitter involved and/or the individual user, and is configured according to algorithms generated from data from other individuals or populations, and/or is generated/altered or adapted by processes including artificial intelligence and/or machine learning. Preferably, the algorithm provides a varying pressure waves output from the emitters at the focused site in the ear to affect either a sound to be appreciated by the user, or a sound to be reduced or prevented from being heard by the user.

Preferably, the emitter output affects movements of ear structures, such as the ear-drum, by single dimension change in pressure—largely in direction ninety degrees to the plane of the ear-drum, or affects movement by generating three-dimensional waveforms or pressure waves affecting movement configured by the algorithms that may vary in three-dimensional position. Most preferably, this is provided by movement of different aspects of the ear-drum according to pressure changes focused by the emitter at different aspects of the surface of the ear-drum. Most preferably, the three-dimensional waveforms may be configured from three-dimensional movement data derived from subjects with normal hearing in response to sound of different frequencies, amplitudes and/or direction.

Preferably, the ultrasound transducer, emitter and receiver, and combined laser and/or light emitter and receiver, and/or separate ultrasound emitter and receiver detect movement of ear structures, for example any part of the ear-drum complex (comprising the ear-drum and malleus), any of the ossicular bones, and/or round or oval window membranes relative to the position of the earphone. Most preferably, this involves measurement using laser triangulation, optical coherence tomography, laser Doppler vibrometry and/or ultrasound to detect movement of the ear-drum complex. Preferably, the output from the sensors is communicated as an input to a processor that may be within the structure containing the emitters (earphone) or connected by wire or wirelessly in another device. Preferably, the algorithm of the processor detects a change in distance between the sensor and the ear-drum complex, vibrations of any portion of the ear-drum, and/or a change in the three-dimensional shape of the ear-drum complex. Most preferably, the processor generates an output dependent upon the algorithm detecting these changes. Further preferably, the algorithm affects a change in the output of the emitters to interfere with the vibration/movement of the ear-drum to dampen or cancel the movement effected by external sound. Most preferably, the algorithm compares the input signal, encompassing information on the vibration/movement of the ear-drum, to the generating or source audio signal, and affects a change in output of the ultrasound emitters to match expected vibration/movement of the ear-drum in relation to the source audio signal. Preferably, vibration or movement of the ear-drum which is not generating a true sound replication of the input audio is altered to generate a vibration or movement that more accurately matches the source audio signal.

Preferably, the algorithm of the processor affects an output from the earphone dependent on the frequency of vibration, and/or the area, direction and/or amplitude of movement of ear structures detected. Auditory attention of a user to a specific sound causes tensing of the ear-drum on the side of interest by tensing a middle ear muscle (the tensor tympani) to tune the ear-drum to the frequency of the sound of interest. Preferably, the output from the dual ultrasound sensor/emitter relates to the auditory attention of the user, and provides an output of the sound being attended to by the ipsilateral ear (on the same side as the earphone) to a local or remote device (via wired or wireless connection). Preferably, this enables local or remote monitoring and recording of the sounds to which the user is listening (auditory attention), and/or amplification of the sound to the user. Preferably, covert surveillance is provided by monitoring and/or recording speech that a user is listening (attending) to. Preferably, use is provided for humans and animals, enabling the attended to sound of animals (such as dogs in search and rescue or covert monitoring roles) to be transmitted and amplified to a remote user and/or recorded. Preferably, this provides monitoring attention of users to sound output, for example to monitor for inattention or fatigue in occupations such as air traffic controllers or to monitor the attention of a student or worker to an audio output, such as a training lecture. Most preferably, the absence of a predetermined sensor input or output that is expected with attention to an audio input would enable an alarm or other signal to alert the user, other person, or user interface to a reduction in the attention of the user.

Preferably, dual ultrasound sensors/emitters detect any movement of ear structures, including those related to voluntary middle ear muscle movements, eye gaze, eye brow movement, eye closure, facial and/or head movements, eustachian tube function, Doppler signals related to ear structure movement and/or blood flow, other biometric changes or external movements, and this affects a change in connected interfaces. Most preferably, a dual ultrasound emitter and sensor array (for example CMUT transducer) is positioned so as to direct ultrasound signals to, and receive from, the external auditory meatus.

Preferably, ultrasound is emitted from the earphone along the ear-canal to air external to the ear-canal. Preferably, the ultrasound receiver detects reflected ultrasound from structures or objects located external to the ear. Preferably, the processor and algorithm are configured to affect change in an interface, following detecting reflected ultrasound. Further preferably, the algorithm and processor detect reflected ultrasound associated with an object positioned or brought into a position adjacent the external auditory meatus, such as a hand covering an ear. Most preferably, in an audio device, the sound output is switched off when a hand is held against the ear. Most preferably, in this context predetermined hand gestures control specific functions of the earphone and affect changes in interfaces, and this may also be user configurable. Preferably, the algorithm is configurable and programmable to an individual user or to specific sensor(s), application and/or use, in response to specific movements or sensed ultrasound signals.

Preferably, ultrasound, sound or vibration directed to the ear-canal walls comprises propagating sound to the middle and/or inner ear by bone conduction through the ear-canal wall. Preferably, the earphone incorporates ultrasound, sound or vibration emitters directed to the ear-canal walls, which affect bone conduction of sound through the canal wall, and also incorporate other emitters which dampen and/or prevent ear-drum movement and/or vibration by creating a sound barrier, actively cancelling external sound, and/or interfering and dampening ear-drum movements and/or vibrations. Most preferably, a silent acoustic environment is created within the outer ear, providing more efficient and accurate transmission of bone conducted signal to the cochlea, with reduced interference from the transmission of external noise.

Preferably, the apparatus is worn like a hearing aid, earphone, ear plug in or adjacent the ear-canal, including in arms or frames of eyeglasses, headsets (including augmented or virtual reality headsets), or is incorporated into other existing technologies such as earphones and multi-function ear devices, which may include functions such as telephone and entertainment audio play back, microphone for telephone, voice communication and voice commands, accelerometers, pulse oximetry, and/or temperature sensors, or other smart sensors.

Advantageously, the invention provides non-contact transmission of sound signals, and control of cochlear implants which are implanted, without any external connections. Advantageously, the invention provides more accurate transmission of a sound to the ear structures, so that the user can more effectively hear a sound of interest, or block-out unwanted, excessive and/or other non-preferred or undesired sound. The invention provides sound transmission for users with hearing reduction related to conditions such as those affecting the middle ear. The invention also provides for hands-free interfaces with the earphone, and provides wireless connection and interaction with cochlear implants or other prosthetic hearing devices or interfaces. Advantageously, the invention provides generation of audible sound from the earphone for users with middle ear disease by generating cochlea stimulation which is not dependent on an intact ear-drum or other middle ear structures such as the ossicles. Further, the invention does not require line of sight between the earphone emitters and the target of focus, unlike laser driven hearing aids which suffer from obstruction by debris within the ear and wax, and require positioning of a laser relative to a target and a physical prosthetic target—for instance a lens. Advantageously, sound received by a microphone of an earphone may have been naturally attenuated and enhanced by the acoustic qualities of the external ear (pinna) and/or ear-canal, providing a more natural sound input than external microphones of current cochlear implants. Further, an absence of a physical connection between a device positioned against the outer skull, and the cochlear implant or hearing prosthetic reduces the risk of surgical harm or damage, and of infection. Advantageously, the invention provides active noise cancellation by using an ultrasound barrier, by negative interference, to specific sounds detected. Advantageously, the invention replicates three-dimensional movement and wave patterns of a normal ear-drum in response to sounds, which includes replicating changes associated with direction of the incoming sound in relationship to the user's ear, and intentional selective auditory focus causing change in tension of the ear-drum by contraction of the tensor tympani muscle. Advantageously, line of sight between emitters and ear-drum is not required, and the ultrasound signal is not affected by debris or wax within the ear-canal. Ultrasound waves are accurately received at the ear-drum and not attenuated by requiring a sealed ear-canal. As such, the earphone operates without requiring the ear-canal to be obstructed, and this allows greater choice of shape and ‘open’ structures, which previously would not work. Further, feedback is reduced, ventilation is improved and comfort, when compared to many offerings presently on the market. Advantageously, the invention provides bone conducting hearing aids and audio devices that are not limited by requiring good bone contact external to the ear. Advantageously, the invention provides control of user interfaces with hand gestures made close to the ear. Advantageously, propagating sound to the middle and/or inner ear by bone conduction through the ear-canal wall enables efficient transmission of the ultrasound to the bone, in a convenient and user acceptable device (such as an earphone) and provides accurate transmission of sound. Further, sound cancellation or blocking is an added advantage.

As used herein, the term ‘adverse pressure wave’ will be understood by those skilled in the art to cover any noise, sound or pressure which may be potentially harmful to the hearing of a user, and/or be an un-preferred or undesired sound or vibration, the transmittal of which is itself un-preferred or undesired.

As used herein, the term ‘ear-drum complex’ will be understood by those skilled in the art to mean the ear-drum and the malleus bone.

### BRIEF DESCRIPTION OF THE DRAWINGS

The invention will now be disclosed, by way of example only, with reference to the following drawings, in which:

FIG. 1 is a pictorial representation of a cross section of the right ear canal and partial view of middle ear showing an emitter in the ear-canal in relation to ear-drum, malleus and tensor tympani muscle;

FIG. 2 is a pictorial representation of the emitter worn in an earphone in the right ear-canal;

FIG. 3 is a graphical representation of an embodiment in which an ultrasound array provides a perception of sound by effecting movement of the ear-drum;

FIG. 4 is a graphical representation of an embodiment in which an ultrasound array generates a sound barrier to reduce or block the transmission of external sound to an ear-drum;

FIG. 5 is a flow chart of an embodiment in line with FIG. 3 in which the ultrasound array provides a perception of sound by effecting movement of the ear-drum;

FIG. 6 is a flow chart of an embodiment in line with FIG. 4 in which the ultrasound array generates a sound barrier to reduce or bock the transmission of external sound to an ear-drum;

FIG. 7 is a graphical representation of an embodiment in which an ultrasound array provides a perception of sound by transmitting signals to a cochlear implant or prosthesis;

FIGS. 8 to 10 are graphical representations of further embodiments in which an ultrasound array provides a perception of sound by transmitting signals to a cochlear implant or prosthesis.

### DETAILED DESCRIPTION OF THE PREFERRED EMBODIMENTS

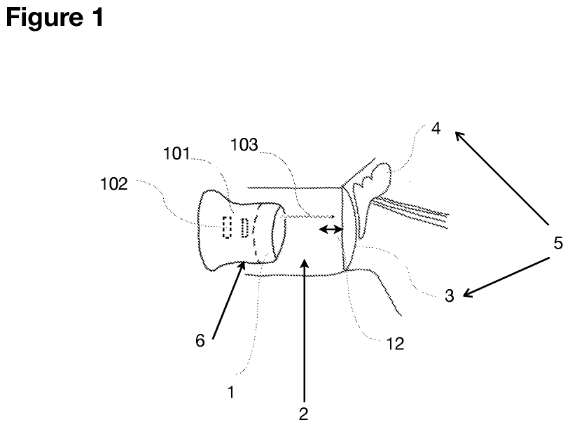

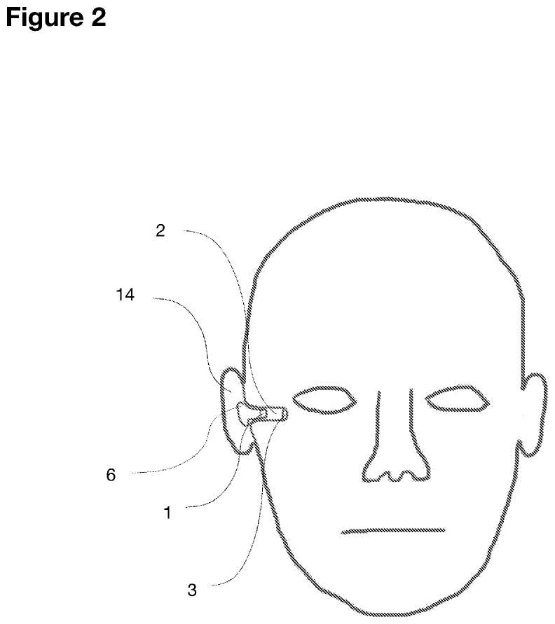

FIGS. 1 and 2 illustrates a first embodiment of apparatus for affecting pressure waves within at least part of or in proximity to an ear of a user. Such an apparatus could be an earphone, hearing aid or ear plug; however, for convenience, just the term earphone will be used.

The earphone 6 is shown positioned (worn) partially within an ear-canal 2 of the user, with an external aspect being located within an external ear (pinna) 14. The earphone 6 includes an ultrasound emitter 1, a processor 101, and a power source (not shown). The emitter 1, which could be an array of emitters, is located on an inward-facing surface of the earphone, and is locatable in the ear-canal 2 such that it is capable of directing ultrasound waves 103 towards an ear-drum complex 5, which includes the ear-drum (tympanic membrane) 3 and the malleus (middle ear bone) 4, which is connected to an inner aspect of the ear-drum 3. The emitter 1 is capable of affecting movement and/or vibration 12 of the ear-drum complex 5, which is transmitted through the malleus 4 to the other interconnected ossicles (bones), and to a cochlea (not shown).

The earphone 6 may include one or more sensors or inputs 102, for providing a control signal or a sound input, and/or be a detector such as a microphone, for detecting sound or changes in pressure at or near the ear of the user. The sensor 102 may be located internally or externally with respect to the user when worn, depending upon its intended use. The emitter 1 may be a standalone emitter or part of an ultrasound transducer (for example a capacitive micro-machined ultrasonic transducer (CMUT)), which has an inbuilt ultrasound receiver. As an alternative, or in addition, the sensor could be an imager, which may be a video camera or an infrared video camera, or have a laser emitting and receiving combination which may have a static or scanning laser element (including laser Doppler vibrometry, optical coherence tomography, or laser triangulation), or be a LIDAR sensor, or a combination of these.

In the above example the earphone 6 is located partially within and partially external to the ear; however, the whole earphone 6 may be located within the ear-canal 2 or, alternatively, the earphone 6 may be located within or adjacent the external ear 14, either as an individual structure or physically connected to a similar structure adjacent the other ear of the user. 14. Although shown directed towards the ear-drum complex 5, the emitter 1 may be located on any surface of the earphone 6, including any combination of any surfaces, such that ultrasound can be emitted towards the ear-drum 3, towards any aspect of the ear-canal 2, radially towards or away from the ear-canal 2, externally along the ear-canal 2 towards an external auditory meatus (external opening of the ear-canal) 2 of the external ear 14.

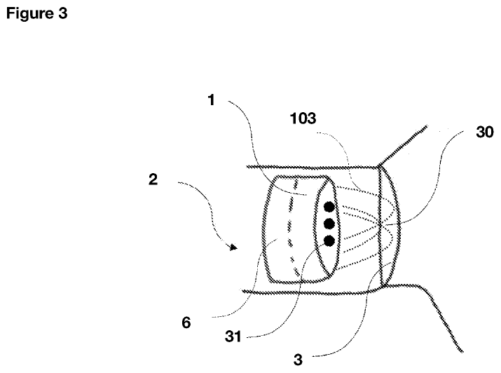

FIG. 3 shows an embodiment in which the earphone 6, as described above in relation to FIGS. 1 and 2, is fully-located in the ear-canal 2 of the user, such that the emitter is directed towards the ear-drum 3. For convenience, and where possible, common features have been given common references. The emitter 1 is provided by an ultrasound array 31 (such as a CMUT transducer) and is capable of emitting ultrasound 103 from emitters configured to interfere 30 at a distance close to the ear-drum 3. In use, the ultrasound array 31 provides a fluctuating pressure wave which generates vibration of the ear-drum 3 which is similar to or the same as vibrations expected in a normal ear in response to a sound input, or that detected by a microphone. The vibrations are transmitted to the cochlea of the user and heard by the user of the device as a perceived sound which is an accurate representation of the sound provided by the sensor or input 102.

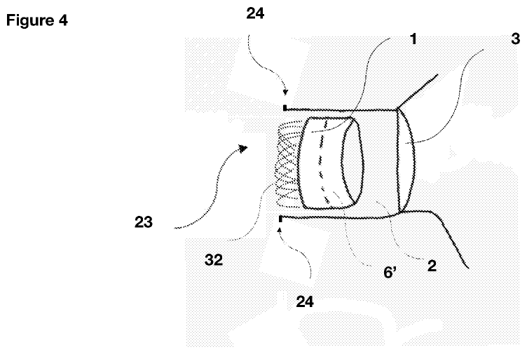

FIG. 4 shows an embodiment in which an ultrasound emitter 1 (such as a CMUT transducer) is located on an outward-facing surface of the earphone 6′, facing oppositely along the ear-canal 2 when compared with the embodiment of FIG. 3. For convenience, and where possible, common features have been given common references. The embodiment of FIG. 4 differs from that of FIG. 3 in that the ultrasound is directed outwards, i.e. away from the ear-drum 3. Specifically, the emitter 1 is capable of directing ultrasound waves 32 towards an external opening (external auditory meatus) 24 of the ear-canal 2. In use, the emitter 1 causes interference of emitted ultrasound waves 32 at a distance close to the external auditory meatus 24 to affect a constant pressure wave 23 across the external opening 24, which prevents, or at least reduces, transmission of external sound to the ear-drum 3. This embodiment seeks to protect a user's hearing in noisy environments by reducing or preventing external sounds—such as overly loud sounds or adverse pressure waves—reaching the ear-drum 3. As such, transmitted audible sound signals generated by audio or ultrasound emitters are heard by the user without interference from background noise or sounds.

The pressure waves 23 may have a differential effect on the transmission of different frequencies or amplitudes of external sounds. For example, the pressure waves 23 may act as high-, low- and/or band-pass filters, and/or act as ultrasonic-, acoustic- and/or diffraction-gratings, reducing pass-through to, and/or deflecting sound waves from, the ear-canal 2 and ear-drum 3. In real terms, this may allow the user to hear speech sound frequencies whilst attenuating lower frequency sounds such as background noise in an aeroplane during flight.

In an alternative embodiment, the earphone 6′ of FIG. 4 may include a microphone for picking up sounds to be transmitted to the ear-drum, and/or for helping configure the sound barrier, noise cancellation and/or pass through functions.

Further, although in the above embodiment the emitter is located so as to face outwardly of the ear, ultrasound emitters may be located on internal walls of an aperture, or channel, provided within the earphone. In this example, the shape of the earphone may equate to an annulus (or hollowed cylindrical body), with emitters directing ultrasound radially inwards to a centre—although outwards is equally possible. The emitters are configured to cause interference of emitted ultrasound waves within the aperture or channel to provide a constant or varying pressure wave within the aperture, or channel. In this example, the earphone does not itself block the ear-canal and may allow sound to reach the ear-drum; however, which sound and how much may be chosen through using the ultrasound waves to reduce transmission of chosen sound by causing a barrier to undesired sound or pressure waves.

Although not shown, an embodiment of the invention includes an earphone 6 having the combined functionality of the examples described in relation to FIGS. 3 and 4.

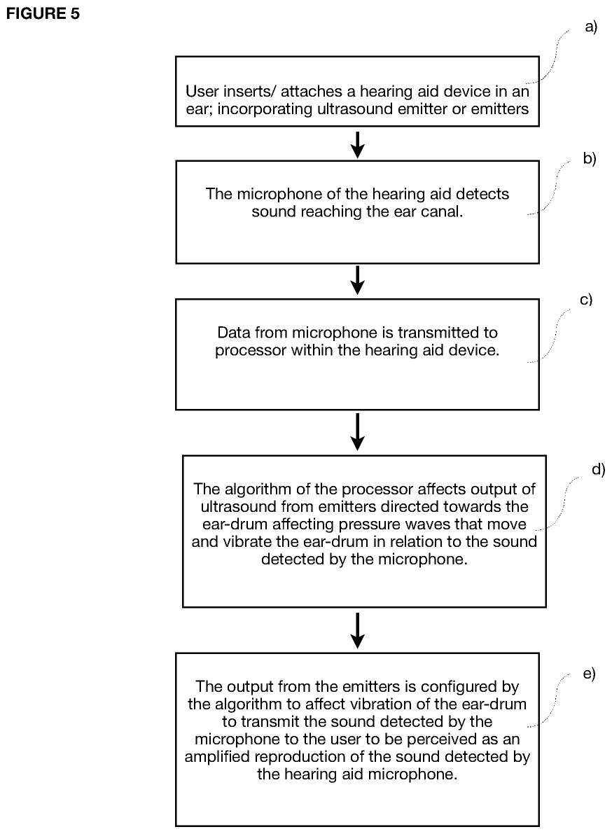

FIG. 5 is a flow chart exemplifying use of the earphone according to FIGS. 1 and 2, or 3, when used as a hearing aid. In this example, the earphone 6 includes a microphone 102 located on an outward-facing surface thereof, for detecting sound received at or near the ear-canal, and the emitter 1 is directed to the ear-drum 3. The following steps are then undertaken:
a) the user inserts the earphone 6 into his or her ear;
b) once the earphone 6 is activated, the microphone 102 detects sound received and provides an output data signal;
c) the output data signal from the microphone 102 provides an input to the processor 101, which input varies in relationship to the frequency and/or amplitude of the sound detected;
d) an algorithm of the processor 101 analyses the received data signal and generates a processor output for activating the ultrasound emitter which output corresponds to the sound received; and
e) the emitter receives the processor output and emits ultrasound waves 103 which affect vibration of the ear-drum to a configured amplitude and frequency consistent with a user hearing the received sound detected by the microphone 102.

In this example the algorithm and processor 101 are configurable for an individual user to amplify preferentially the frequencies which the user is less able to hear. Sound received and detected by the microphone 102 is amplified to enable the user to better hear and understand external sound of interest, such as speech.

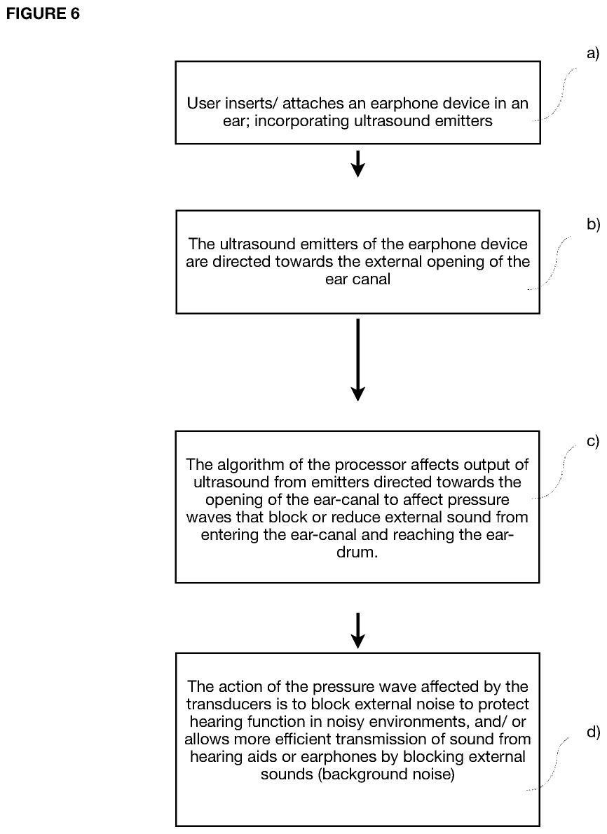

FIG. 6 is a flow chart exemplifying use of the earphone 6′ according to FIG. 4, when used as a hearing protector. In this example, the emitter 1 is located on the outward-facing surface of the earphone 6′ such that ultrasound waves are directed towards the external opening 24. The following steps are undertaken:
a) the user inserts the earphone 6 into his or her ear;
b) the emitter 1 is directed towards the external opening 24 of the ear-canal 2;
c) once activated, the algorithm of the processor 101 generates a processor output for activating the ultrasound emitter 1; and
d) the emitter 1 receives the processor output and emits ultrasound waves 32 which are configured to generate ultrasound interference 23 to affect air pressure waves at or near the external opening 24 of the ear-canal 2, which block or reduce the amount of external sound entering the ear-canal 2 and reaching the ear-drum 3.

In one example, a constant pressure wave acts as a barrier to external sound propagating along the ear-canal, and reduces the amount of external noise heard by the user. In a further example, the pressure waves act as acoustic filters or acoustic grates, affecting transmission of sound to the ear-drum. This embodiment enables protection of the inner ear structures against loud and/or constant noise which may damage the cochlea, and enables output sound from hearing aids and other earphones to be heard more clearly with reduced interference from background noise.

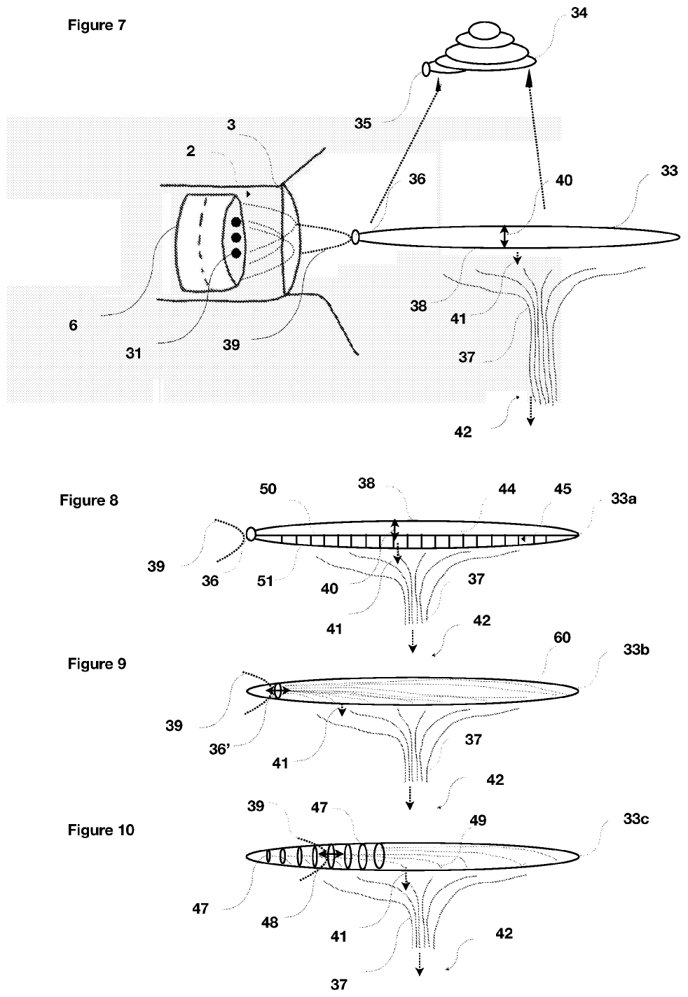

FIG. 7 shows an embodiment of earphone based upon the one described in relation to FIG. 3. The earphone 6 is shown located in the ear-canal 2 of a user, such that ultrasound waves can be directed towards the ear-drum 3 of the user. The earphone 6 includes an ultrasound array 31 (such as a CMUT transducer). The user suffers from a form of hearing difficulty and has a cochlear implant 33 implanted within its cochlea 34, to lie within the spiral structure of the cochlear canal. In FIG. 7 the cochlear implant 33 is for demonstration purposes shown as a linear structure, but would have a shape to correspond with the cochlea 34. FIG. 7 also demonstrates how the cochlear implant 33 stimulates auditory nerve cells 37 of the inner ear (including spiral ganglion neurones). The structure of the cochlea 34 is shown with its oval window 35, which in some versions may remain intact, or be replaced by a receiver 36, for receiving ultrasound or sound waves, attached to the cochlear implant 33. The cochlear implant 33 may be positioned with the receiver 36 facing outwards, for example located at a position of the oval window 35 or round window, or within a surgically formed canal in continuity with the cochlea 34. The function of the receiver 36 is to propagate audio, ultrasound waves or electromagnetic signals to the cochlear implant 33. As shown, the cochlear implant 33 is a longitudinal device forming a chamber 38, which may contain fluid of a determined viscosity. An outer aspect of the chamber 38 is located abutting and/or close to the nerve cells 37, and is provided by electrical conduction material (electrodes) and/or separate or continuous piezoelectric materials, which may be superficial and/or extend into the structure of the cochlear implant 33. The cochlear implant 33 may be composed, at least partially, of piezoelectric or similar material which is stimulated to produce electrical current by the action of the ultrasound waves.

A distance along the cochlear implant 33 where electrical current 41 is generated is determined by the characteristics of the ultrasound wave 39 emitted—determined by the frequency, amplitude and/or the modulation of the ultrasound—from ultrasound emitters 31 on the earphone 6 located within the ear-canal 2, or, alternatively, from an ultrasound emitter or emitters which are positioned external to the ear-canal, for example behind the ear or on the scalp.

In one example of use, ultrasound waves 39 from the emitter 31 affect pressure changes/waves 40 within the material or substance of the cochlear implant 33. The pressure waves 40 affect movement of an aspect of the cochlear implant at a distance along the cochlear canal 40. The movement generates an electrical current 41 within the electrodes or piezoelectric material, which are adjacent the nerve cells 37 and, thereby, generates signals in an auditory nerve 42 of the user, such that those signals are perceived by the user as sound. The distance along the cochlear implant 33 where the waves 40 generate the movement determines the frequency of the sound heard by the user.

In a further example of use, a microphone 102 of the earphone 6 receives sound and sends audio signals to a processor 101, which analyses the audio signals, and an algorithm of the processor 101 causes the ultrasound emitter 31 to emit ultrasound waves from multiple emitters, which interfere at a location within or close to the cochlear implant 33, so as to generate vibration of the receiver 36 at the same sound wave frequencies as the received sound.

In a further example of use, ultrasound waves 39 from multiple emitters 1; 31 interfere to generate pressure waves 40 in the substance or material of the cochlear implant 33, at distances along the cochlear implant 33 related to the audio input frequency.

In a further example of use, interference patterns of ultrasound waves 39 emitted from the emitters 31 directly affect a distance at which the pressure waves 40 are generated along the cochlear implant 33 according to the audio input.

The invention provides non-contact transmission of sound signals, and control of cochlear implants which are implanted without any external connections.

FIGS. 8, 9 and 10 show different embodiments of cochlear implant, which are each implanted or located as describe in relation to FIG. 7. As such, it is only the differences and main features of these different embodiments which will be discussed.

FIG. 8 shows an embodiment which builds upon that described in relation to FIG. 7. The cochlear implant 33a is a fluid filled, longitudinal structure which is locatable in a spiral configuration within the cochlear canal. The implant 33a has an internal structure of one or multiple separate longitudinal chambers 38, which may contain fluid of similar or different viscosity. Piezoelectric material 45 extends into the structure of the cochlear implant 33, such that one end is connected to a central longitudinal structure 50 and the other end to an outer surface 51 of the implant 33a. Multiple separate piezoelectric structures are fixed similarly along the length of the cochlear implant 33a, and are affected to move by pressure waves 40 within the chamber 38 caused by the ultrasound.

FIG. 9 shows an embodiment which builds upon that described in relation to FIG. 7. The cochlear implant 33b—which is locatable in a spiral configuration within the cochlear canal—includes a receiver 36′ which is provided by piezoelectric material, or similar, and includes one or more conducting structures 60 that may filter the electrical current generated by the receiver 36′, which receiver moves and/or vibrates upon receipt of the ultrasound waves 39. The conducting structures 60 are electrical conductors, for example wires, located within the structure/material of the implant 33b. The conducting structures 60 provide electrical current 41 from the implant 33b to the nerve cells 37 but according to a frequency of the electrical activity generated by the receiver 36′. For example, a lower frequency of sound detected by a microphone 102 is configured to generate ultrasound generated movement in the receiver 36′ at the same frequency as the detected sound and the filter affects transmission of electrical activity to the electrodes at a location further along the cochlear implant 33b from the receiver 36′ than for a higher frequency sound.

FIG. 10 shows an embodiment which builds upon FIG. 9. The cochlear implant 33c—which is locatable in a spiral configuration within the cochlear canal—includes multiple outwardly facing receiving surfaces 47, or structures, at different positions along the length of the longitudinal structure of the cochlear implant 33c. The receiving surfaces 47, or structures, include piezoelectric materials, or similar materials, that generate electrical current in response to movement 48, or indeed other physical properties generated by the effect of audible sound, pressure waves, and/or ultrasound waves on the surfaces 47. Each of the receiving surfaces 47 is connected by electrical conducting material 49 (such as a wire) to an electrode or electrodes at a different location along the longitudinal structure of the cochlear implant 33c so as to generate electrical current 41 dependent upon the ultrasound waves 39 and, thereby, a control input, audio input or detected sound input. Accordingly, this embodiment provides a perception of sound to the user.

1. An apparatus for affecting pressure waves within at least part of or in proximity to an ear of a user, at least part of the apparatus being configured to be wearable within at least part of or in proximity to said ear of said user,
the apparatus further comprises processor means operative in response to an input signal of an input means to control an output of an ultrasound emitter,
wherein the ultrasound emitter is configured to generate an ultrasound output which affects air and/or biological tissue in proximity to the ultrasound emitter.

2. The apparatus as claimed in claim 1, wherein the ultrasound output is configured to be capable of transmitting, either directly or indirectly, ultrasound towards one or more of the group comprising:
a) at least part of:
i) an ear-drum complex to affect movement and/or vibration of at least part of the ear-drum complex; or
ii) a inner and/or middle ear structure or space(s) to affect movement and/or vibration of at least part of an inner and/or middle ear structure or space(s);
b) at least part of an ear canal, to affect movement and/or vibration of at least part of an ear-canal wall, adjacent bone and/or volume of air in the ear-canal;
c) at least part of a cochlear implant or hearing prosthetic, to affect movement and/or vibration of at least part of the cochlear implant or hearing prosthetic;
d) at least part of an external opening of an/the ear canal,
e) a region adjacent an outer ear of said user; and/or
f) a region adjacent, or radially inwards of, the apparatus.

3. The apparatus as claimed in claim 1, wherein the ultrasound output generates a fluctuating pressure wave of variable frequency and/or amplitude configured to replicate vibration of an ear-drum commensurate with vibrations expected in a normal ear in response to sound.

4. The apparatus as claimed in claim 1, wherein the ultrasound output generates:
a) a pressure effect at or within, or in proximity to an ear, ear-drum and/or ear-canal;
b) patterns of ultrasound waves that interfere at one or more points, areas, and/or two- or three-dimensional planes to affect a change in pressure at or close to any of the points, areas or planes;
c) patterns of ultrasound waves that interfere at a position or area, in or adjacent to a site or position within the ear-canal, the external auditory meatus, and/or the ear-canal wall and/or surrounding bone;
d) a constant or fluctuating pressure wave of constant or varying frequency and/or amplitude configured to provide interference to reduce or obstruct the effects of an adverse pressure wave reaching said ear-drum complex, cochlear implant or hearing prosthetic;
e) a constant pressure wave configured to act as an acoustic filter or acoustic grate, to reduce or prevent transmission of specific frequencies and/or amplitudes of an adverse pressure wave reaching said ear-drum complex, cochlear implant or hearing prosthetic; and/or
f) patterns of ultrasound waves to provide interference to reduce or obstruct the effects of an adverse pressure wave reaching said ear-drum complex, cochlear implant or hearing prosthetic, or modify an adverse pressure wave to remove its adverse effect(s).

5. The apparatus as claimed in claim 1, wherein the input means comprises:
a control signal;
a sound input; and/or
a detector, for detecting sound or changes in pressure at or near said ear of said user.

6. The apparatus as claimed in claim 5, wherein the input signal to the processor means is analysed to determine frequencies and corresponding amplitudes of the sound input signal and/or detected sound signal, and the processor means provides an output signal for the emitter means which is configured to affect:
at least part of the cochlear implant or hearing prosthetic to generate signals in the auditory nerve which are perceived by said user as audible sound; or
the ear-drum complex consistent with said user hearing the sound input or detected sound.

7. The apparatus as claimed in claim 1, wherein the processor means is configured to provide an output signal to the ultrasound emitter for providing ultrasound of predetermined amplitude, frequency, duration, modulation, harmonics, timing, and/or interference pattern.

8. The apparatus as claimed in claim 6, wherein the processor means is configured to additionally analyse one or more inputs from an ultrasound transducer/receiver, a laser and/or light emitter/receiver, microphone, proximity sensor, a time of flight sensor, an imager, and/or an ultrasound transducer/receiver, or combination thereof, which detect movement and/or vibration of any ear structure, or a movement in close proximity to said ear, and, thereby, modify the output signal.

9. The apparatus as claimed in claim 1, wherein the processor means is configured to compare a detected response of one or more ear structures to the ultrasound output with an expected response to sound, and is configured to modify the ultrasound output to improve replication of the sound.

10. The apparatus as claimed in claim 1 further comprising a cochlear apparatus.

11. A method for affecting pressure waves within at least part of or in proximity to an ear of a user, the method comprises controlling an output of an ultrasound emitter in response to an input signal of an input means,
wherein the ultrasound emitter is worn within at least part of or in proximity to the ear of the user and generates an ultrasound output which affects air and/or biological tissue of the user in proximity to the ultrasound emitter.

12. The method as claimed in claim 11, wherein the ultrasound output transmits, either directly or indirectly, ultrasound towards one or more of the group comprising:
a) at least part of
i) an ear-drum complex of the user to affect movement and/or vibration of at least part of the ear-drum complex; or
ii) an inner and/or middle ear structure or space(s) to affect movement and/or vibration of at least part of an inner and/or middle ear structure or space(s);
b) at least part of an ear-canal of the user, to affect movement and/or vibration of at least part of an ear-canal wall, adjacent bone and/or volume of air in the ear-canal;
c) at least part of a cochlear implant or hearing prosthetic of the user, to affect movement and/or vibration of at least part of the cochlear implant or hearing prosthetic;
d) at least part of an external opening of an/the ear canal,
e) a region adjacent an outer ear of the user; and/or
f) a region adjacent, or radially inwards of, an associated apparatus.

13. The method as claimed in claim 12, wherein the ultrasound output transmits to at least part of the ear-drum complex, inner or middle ear, ear-canal or other structure or space, or to at least part of the cochlear implant or hearing prosthetic to provide a perception of sound to the user.

14. The method as claimed in claim 12, wherein the ultrasound output transmits to at least part of the ear canal, the region adjacent the outer ear of the user, or the region adjacent, or radially inwards of, the associated apparatus to reduce or obstruct the effects of an adverse pressure wave reaching said ear-drum complex, cochlear implant or hearing prosthetic.

15. The method as claimed in claim 11, wherein the ultrasound output generates a fluctuating pressure wave of variable frequency and/or amplitude for replicating vibration of an/the ear-drum commensurate with vibrations expected in a normal ear in response to sound.

16. The method as claimed in claim 11, wherein the ultrasound output generates:
a) a pressure effect at or within, or in proximity to an ear, ear-drum and/or ear canal;
b) patterns of ultrasound waves that interfere at one or more points, areas and/or two- or three-dimensional planes to affect a change in pressure at or close to any of the points, areas or planes;
c) patterns of ultrasound waves that interfere at a position or area, in or adjacent to a site or position within the ear-canal, the external auditory meatus, and/or the ear-canal wall and/or surrounding bone;
d) a constant or fluctuating pressure wave of constant or varying frequency and/or amplitude for providing interference to reduce or obstruct the effects of an/the adverse pressure wave reaching said ear-drum complex, cochlear implant or hearing prosthetic;
e) a constant pressure wave for acting as an acoustic filter or acoustic grate, to reduce or prevent transmission of specific frequencies and/or amplitudes of an adverse pressure wave reaching said ear-drum complex, cochlear implant or hearing prosthetic; and/or
f) patterns of ultrasound waves for providing interference to reduce or obstruct the effects of an adverse pressure wave reaching said ear-drum complex, cochlear implant or hearing prosthetic, or modify an adverse pressure wave to remove its adverse effects.

17. The method as claimed in claim 11, wherein an input signal to a processor means is analysed to determine frequencies and corresponding amplitudes of a sound input signal and/or detected sound signal, and the processor means provides an output signal for the emitter means which is configured to affect:
at least part of the cochlear implant or hearing prosthetic to generate signals in the auditory nerve which are perceived by the user as audible sound; or
the ear-drum complex consistent with the user hearing the sound input or detected sound.

18. The method as claimed in claim 11 comprises processing an input signal to compare a detected response of one or more ear structures to the ultrasound output with an expected response to sound, and modifying the ultrasound output to improve replication of the sound.

19. A cochlear apparatus, for implantation within at least part of a cochlear canal of a user or for replacement of a cochlea of a user, the apparatus comprises one or more regions adapted to receive an ultrasound output of an ultrasound emitter locatable within at least part of or in proximity to an ear of said user so as to generate one or more electrical signals for providing a perception of sound to said user.

20. The cochlear apparatus as claimed in claim 19, wherein the apparatus comprises means for stimulating auditory nerve cells of the inner ear.

21. The cochlear apparatus as claimed in claim 19, wherein the one or more regions comprise piezoelectric material capable of receiving said ultrasound output and generating one or more electrical signals.

22. The cochlear apparatus as claimed in claim 19, wherein the one or more regions are spatially located along at least part of the apparatus such that different regions are responsive to different frequencies, amplitudes and/or modulation of said ultrasound output.

23. The cochlear apparatus as claimed in claim 19, wherein the apparatus comprises a receiving surface for propagating received sound, ultrasound or electromagnetic signals to the cochlear apparatus.
